(19) United States
(12) Patent Application Publication
(10) Pub. No.: US 2014/0094681 A1
(43) Pub. Date: Apr. 3, 2014

(54) SYSTEM FOR NAVIGATING SURGICAL INSTRUMENTS ADJACENT TISSUE OF INTEREST

(71) Applicant: COVIDIEN LP, Mansfield, MA (US)

(72) Inventors: CHRISTOPHER A. VALENTINE, BOULDER, CO (US); KRISTIN D. JOHNSON, LOUISVILLE, CO (US); BENJAMIN M. CORUM, BOULDER, CO (US); SCOTT F. ALEXANDER, WESTMINSTER, CO (US); CHRISTOPHER T. RUSIN, GOLDEN, CO (US)

(73) Assignee: COVIDIEN LP, Mansfield, MA (US)

(21) Appl. No.: 14/036,594

(22) Filed: Sep. 25, 2013

Related U.S. Application Data
(60) Provisional application No. 61/709,004, filed on Oct. 2, 2012.

(51) Int. Cl. A61B 5/06; A61B 1/00; A61F 2/00; A61M 5/00; A61B 5/05; A61B 5/00; A61B 17/068; A61B 17/115; A61B 17/22; A61B 17/00; A61B 17/11; A61B 17/08; A61B 17/06; A61F 2/82; A61B 17/064; A61B 19/00

(52) U.S. Cl. CPC A61B 5/062; A61B 19/20; A61B 19/5244; A61B 1/00158; A61F 2/0063; A61M 5/007; A61B 5/05; A61B 5/4836; A61B 5/7405; A61B 5/742; A61B 19/2203; A61B 17/068; A61B 17/115; A61B 17/22; A61B 17/00234; A61B 17/1114; A61B 17/083; A61B 17/06166; A61F 2/82; A61B 17/064
USPC 600/409

(57) ABSTRACT

A system for navigating a surgical instrument adjacent tissue of interest is provided and includes a magnetic device and a surgical instrument. The magnetic device is positionable within a body of a patient. The surgical instrument includes a magnetic detection device that is operably disposed thereon to detect the magnetic device. The surgical instrument is configured to remove tissue of interest within the patient.

### CROSS REFERENCE TO RELATED APPLICATION

The present application claims the benefit of and priority to U.S. Provisional Application Ser. No. 61/709,004, filed on Oct. 2, 2012, the entire contents of which are incorporated herein by reference.

### BACKGROUND

1. Technical Field

The present disclosure relates to a system for navigating surgical instruments adjacent tissue of interest. More particularly, the present disclosure relates to systems that utilize magnetic signals to locate diseased tissue.

2. Description of Related Art

Systems and methods that are utilized to locate and remove diseased tissue are well known in the art. For example, a partial bowel resection may be carried out to remove adenomas or other suspicious or diseased tissue on the colon. In certain instances, the tissue is identified by colonoscopy but judged not amenable to removal with a colonoscope, e.g., where the diseased tissue is quite large. For example, in the instance where the diseased tissue is 7-10 inches in length, the colon may be resected (i.e., the diseased tissue is located in the middle of a specimen that is to be removed). Thereafter, the remaining ends of the bowel may be rejoined.

Typically, the resection is performed laparoscopically after the diseased tissue's location is marked with India ink tattoos via the colonoscope. The India ink indicates to a surgeon where the diseased tissue is located and facilitates with the resection. On occasion, however, the India ink may be difficult or even impossible to locate, which, in turn, may make the resection difficult or impossible to carry out.

To overcome the aforementioned drawbacks associated with India ink, endoscopic clips have been employed to mark the diseased tissue. The clips, however, in some instances are difficult or otherwise impossible to find due to difficulty in palpating the endoscopic clip, which are, typically quite small.

### SUMMARY

In view of the foregoing, systems that utilize magnetic signals to navigate surgical instruments adjacent diseased tissue may prove useful it the medical art.

Embodiments of the present disclosure are described in detail with reference to the drawing figures wherein like reference numerals identify similar or identical elements. As used herein, the term “distal” refers to a portion of a surgical instrument that is being described which is further from a user, while the term “proximal” refers to the portion of the surgical instrument that is being described which is closer to a user.

An aspect of the present disclosure provides a system for navigating a surgical instrument adjacent tissue of interest. The system includes a magnetic device that is positionable within a body of a patient. A surgical instrument includes a magnetic detection device that is operably disposed thereon to detect the magnetic device. The surgical instrument is configured to remove tissue of interest within the patient. The magnetic device may be positioned within the body of a patient via one of an endoscope and being ingested. The tissue of interest may be located on a bowel, liver, lung or fibroids. The magnetic device may be configured to penetrate through a mucosa and into a submucosal space of a bowel wall to prevent migration of the magnetic device. The magnetic detection device may be configured to create software coupled imaging information to a monitor in real-time. The surgical instrument may be a resection device.

The magnetic device may be a magnetic element including but not limited to a clip and tag, stent, staple, silvered sutures, injectable solution including a viscous base material with magnetic particles, and powered chip configured to emit one of an electromagnetic and magnetic field. The injectable solution including a viscous base material with magnetic particles may be an India ink with one of magnetic and ferrous particles suspended therein.

The magnetic device may be operably coupled to a surgical mesh that includes a helical tack thereon for attaching the surgical mesh including the magnetic device to tissue adjacent the tissue of interest.

The powered chip may be either an active RFID device or a passive RFID device. In this instance, the magnetic detection device on the surgical instrument may include one or more RFID detectors thereon that are configured to detect the active or passive RFID devices.

The magnetic detection device on the surgical instrument may include two or more coils that are positioned in close proximity to one another and are rigidly fixed with respect to one another. In this instance, and in a non-magnetic media, the two or more coils have a stable mutual coupling of their magnetic fields. Moreover, when the two or more coils are positioned adjacent the magnetic device, the mutual coupling of their magnetic fields is substantially changed and, subsequently, one or more indications may be provided to an end user. The one or more indications may include an audio or a visual indicator. Alternatively, the magnetic detection device on the surgical instrument may include a single coil. When the coil is positioned adjacent the magnetic device, the indication(s) may be provided to an end user.

A robotic arm having a sensor thereon may be configured to produce a signal as a result of the sensor coming into proximity of the magnetic device. The signal being representative of the position of the magnetic device with respect to the sensor.

According to another aspect of the present disclosure, a system for navigating a surgical instrument adjacent tissue of interest is provided. The system includes one or more magnetic devices that are positionable within a body of a patient and attachable to tissue adjacent the tissue of interest. The magnetic device is configured to emit a magnetic field. A surgical instrument includes a magnetic detection device that is operably disposed thereon and is configured to detect the magnetic field emitted by the magnetic device. The surgical instrument is configured to remove tissue of interest within the patient. A monitor in operable communication with the magnetic device and surgical instrument is configured to provide a visual or audio indication of the proximity of the surgical instrument with respect to the magnetic device. The magnetic device may be positioned within the body of a patient via either an endoscope or being ingested.

The magnetic device may be a magnetic element including, but not limited to a clip and tag, stent, staple, silvered sutures, injectable solution including a viscous base material with magnetic particles, or a powered chip configured to emit one of an electromagnetic and magnetic field.

The magnetic detection device on the surgical instrument may include two or more coils that are positioned in close proximity to one another and are rigidly fixed with respect to one another. In this instance, and in a non-magnetic media, the two or more coils have a stable mutual coupling of their magnetic fields. When the two or more coils are positioned adjacent the magnetic device the mutual coupling of their magnetic fields is substantially changed and, subsequently, one or more indications may be provided to the monitor. The one or more indications may include an audio or a visual indicator. Alternatively, the magnetic detection device on the surgical instrument may include a single coil. When the coil is positioned adjacent the magnetic device, the indication(s) may be provided to an end user.

### BRIEF DESCRIPTION OF THE DRAWING

Various embodiments of the present disclosure are described hereinbelow with references to the drawings, wherein:

FIG. 1 is a schematic block diagram illustrating a system according to an embodiment of the present disclosure; and

FIG. 2 is side view of a bowel having a tumor that is to be removed with the system depicted in FIG. 1.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

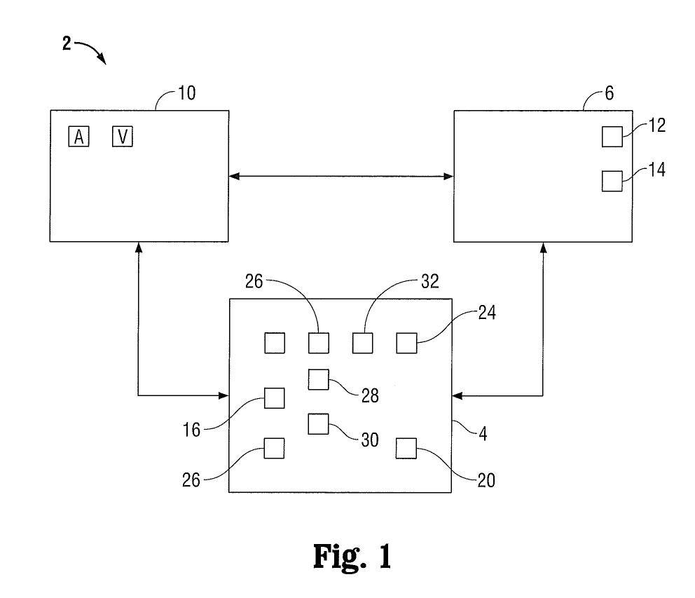

Turning now to FIG. 1, a surgical system 2 according to an embodiment of the present disclosure is illustrated. In the illustrated embodiment, system 2 includes a magnetic device 4 and one or more surgical instruments 6. In embodiments, an optional monitor 10 may be configured to communicate with one or both of magnetic device 4 and surgical instrument(s) 6 (FIG. 1).

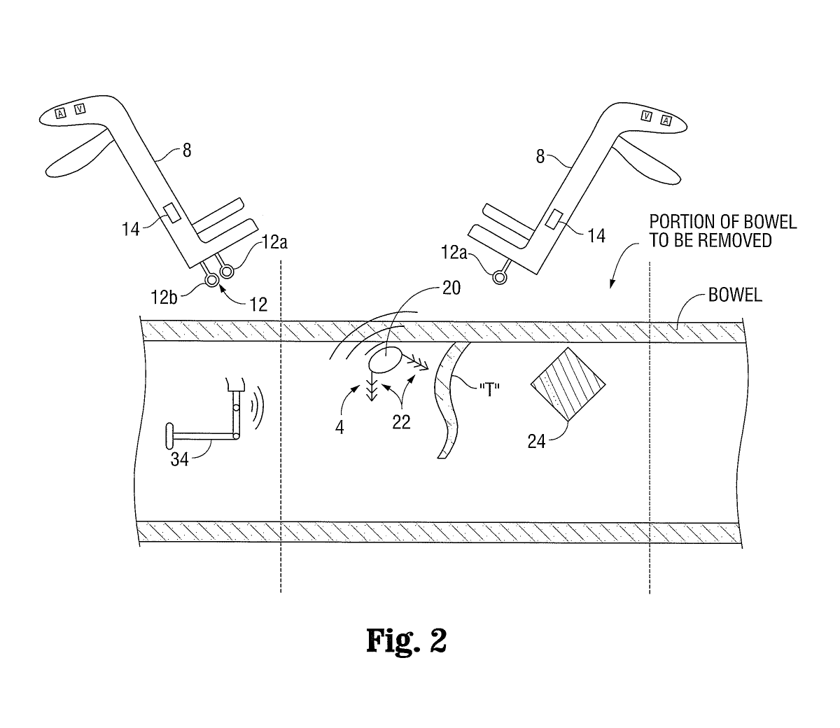

Surgical instrument 6 is configured to interact with tissue. As defined herein, the term interact is not limited to include treat (including electrosurgically treat), remove and capture tissue. Accordingly, surgical instrument 6 may be any suitable surgical instrument configured to perform the aforementioned interactions with tissue. In one particular embodiment, such as the embodiment illustrated in FIG. 2, surgical instrument 6 is a transverse anastomosis stapler 8 (stapler 8), which is known in the art. A suitable stapler 8 may be configured to function in a manner consistent with the teaching of U.S. Pat. No. 7,070,083, the contents of which incorporated in its entirety herein. Other types of surgical instruments that may be utilized in accordance with the instant disclosure include, but are not limited to electrosurgical forceps (of the open or closed type), endoscopic pouches, electrosurgical pencils (of the RF or microwave type), etc.

Surgical instrument 6 is configured to support a magnetic detection device 12 thereon. For the remainder of the disclosure, magnetic detection device 12 is described in terms of use with the stapler 8.

Magnetic detection device 12 is operably supported at a distal end of stapler 8, see FIG. 2. In accordance with the instant disclosure, magnetic detection device 12 may be configured to function similar to a metal detector. In one particular embodiment, for example, magnetic device 12 may include two or more coils 12a and 12b that are positioned at the distal end of the stapler 8. Coils 12a and 12b are disposed in close proximity to one another and are rigidly fixed with respect to one another. Each of coils 12a and 12b is configured to provide its own electromagnetic field. A power supply (not shown) on stapler 8 is configured to generate sufficient current through coils 12a and 12b to produce a respective electromagnetic field therefrom. In an assembled configuration, and in a non-magnetic media (e.g., air, blood, etc.), coils 12a and 12b have a stable mutual coupling of their magnetic fields.

Magnetic detection device 12 is configured to detect one or more types of magnetic fields. The magnetic field may be associated with a ferromagnetic material (e.g., a passive magnetic device) or the magnetic field may be generated by an electromagnet that is configured to provide an alternating current through windings of a conductor to produce an electromagnetic field (e.g., an active magnetic device). In the latter instance, only one of coils 12a and 12b needs to be provided on stapler 8. In accordance with the instant disclosure, coils 12a and 12b are configured to detect a magnetic field emitted from magnetic device 4.

In embodiments, it may prove advantageous to provide a magnetic detection device 12 that includes a powered chip reader (or detector). For example, and in one particular embodiment, an RFID device, e.g., an RFID reader, may be operably supported on the stapler 8, e.g., at the distal end of stapler 8. The RFID reader may be utilized when the magnetic device 4 is a powered chip that is configured to emit an electromagnetic and/or magnetic field, e.g., an RFID tag 16 (e.g., active or passive RFID tags).

Magnetic device 4 provides a magnetic field that is detectable by magnetic detection device 12. To this end, magnetic device 4 may be in the form of, but is not limited to magnetic clips 26, tags 20, meshes 24, stents 26 or staples 28, silvered sutures 30, an injectable solution including a viscous base material with magnetic particles 32 or, as noted above, an RFID tag 16 (FIG. 1). As can be appreciated, other types of magnetic devices not disclosed herein may also be utilized with system 2. The key feature(s) of magnetic device 4 is that it is capable of emitting a magnetic field that is detectable by magnetic detection device 12. More particularly, in the instance where magnetic device 4 is an electromagnetic device, a single coil, e.g., coil 12a, may be positioned on surgical instrument 6 and configured to detect the magnetic field emitted from magnetic device 4. Moreover, in the instance where magnetic device 4 is a ferromagnetic material, both of coils 12a, 12b may be positioned on surgical instrument 6 and configured to detect the magnetic field emitted from magnetic device 4. In this embodiment, when coils 12a, 12b are positioned in a vicinity of the ferromagnetic material, the mutual coupling of their magnetic fields is disrupted (and/or substantially changed) and, subsequently, one or more indications may be provided to an end user, as described in more detail below.

In certain instances, it may prove advantageous to provide magnetic device 4 with tissue penetrating and/or tissue adhering capabilities. For example, magnetic device 4 may be configured to penetrate through (or otherwise adhere to) a mucosa and into a submucosal space of a bowel wall to prevent migration of magnetic device 4 after magnetic device 4 is positioned adjacent tissue of interest “T” (FIG. 2). For example, and in one particular embodiment, magnetic device 4 may be in the form of a magnetic tag 20 having one or more barbed ends 22 (a pair of barbed ends 22 are shown) that are configured to penetrate tissue to affix magnetic tag 20 adjacent to tissue of interest “T” (FIG. 2).

In an embodiment, magnetic device 4 may be in the form of a magnetic mesh 24 that is configured to adhere to tissue (FIG. 2). Mesh 24 may include one or more structures and/or materials operably disposed thereon to facilitate coupling mesh 24 to tissue. For example, mesh 24 may include any number of tacks, barbs and/or adhesives to facilitate coupling the mesh 24 to tissue.

Magnetic device 4 may be positioned adjacent tissue of interest “T” by any suitable methods. For example, and in the embodiment illustrated in FIG. 2, a colonoscope may be utilized to position magnetic device 4 adjacent tissue of interest “T.” Alternately, magnetic device 4 may be ingested; this may prove advantageous when a patient is pregnant or otherwise not amenable to undergo X-rays or imaging. An ingestible magnetic device 4 may be in the form of a viscous base material, e.g., India ink, with magnetic and ferrous particles suspended therein; other viscous based materials are contemplated herein.

In certain instances, one or more indications may be provided to an end user, e.g., an audio “A” and/or a visual “V” indicator, when magnetic detection device 12 is positioned close to magnetic device 4. The audio “A” and/or visual “V” indications may be utilized with any of the aforementioned types of magnetic detection devices 12, e.g., a single coil 12a; coils 12a and 12b; and RFID reader 14. The audio “A” and/or visual “V” indicators may be provided by a stand-alone device, e.g., monitor 10 that may include audio “A” capabilities (FIG. 1), or may be provided by the stapler 8 (FIG. 2). In the former instance, monitor 10 may provide a visual aid to an end user of the proximity of magnetic detection device 12 with respect to magnetic device 4. In this instance, magnetic detection device 12 may be configured to create software-coupled imaging information to monitor 10 in real-time. In the latter instance, stapler 8 may include a speaker (not shown) or may include one or more LEDs (or other visual indicators) thereon to indicate to a user that magnetic detection device 12 is in close proximity to magnetic device 4. In this instance, the speaker and/or LEDs may be positioned near a handle assembly of surgical instrument 6. Moreover, it may prove useful to utilize scaled audio “A” and/or visual “V” indicators which may be configured to change intensity, volume, frequency, etc. proportionally with proximity of magnetic detection device 12 with respect to magnetic device 4.

Operation of system 2 is described in terms of performing a partial bowel resection. For purposes herein, magnetic device 4 is assumed to be magnetic clip 20 that has barbs 22 on it. In use, magnetic clip 20 is positioned adjacent tissue of interest “T” such as, for example, within a lumen of a bowel of a patient (FIG. 2). A colonoscope may be utilized to position magnetic clip 20 within the bowl, see FIG. 2.

Subsequently, stapler 8 is navigated through the patient. In one particular embodiment, stapler 8 communicates with monitor 10. Monitor 10 provides an indication to the user as to the position of stapler 8 with respect to magnetic clip 20.

Once in position, a user can staple and sever tissue on each side of magnetic clip 20 leaving two closed ends of bowel.

Thereafter, and as is common in a partial bowl resection, one or more devices (e.g., a circular anastomosis stapler) may be utilized to join the two closed ends and create a lumen therethrough.

System 2 overcomes the aforementioned drawbacks typically associated with conventional systems utilized to locate and remove diseased tissue. In particular, the issue of India ink being difficult and/or impossible to find is overcome due to the use of magnetic detection device 12 and magnetic device 4. In addition, because magnetic detection device 12 utilizes a magnetic field to detect magnetic device 4, magnetic device 4 does not require palpating.

From the foregoing and with reference to the various figure drawings, those skilled in the art will appreciate that certain modifications can also be made to the present disclosure without departing from the scope of the same. For example, system 2 may utilized with one or more types of robotic devices. For example, magnetic device 4 may be operably coupled to a robotic arm 34 (or the like). In this instance, robotic arm 34 may be utilized to navigate surgical instrument 6 through the bowel and adjacent magnetic device 4.

While the above system 2 has been described herein in terms of use within a controlled surgical environment, it is within the purview of the present disclosure that system 2 may be utilized in non-controlled environments. For example, magnetic detection device 12 may be operably disposed on a wand like device (not shown) and utilized on a field of combat. In this instance, for example, magnetic device 12 may be utilized by a medic to find shrapnel (or other metallic object) that is lodged within a soldier.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for navigating a surgical instrument adjacent tissue of interest, comprising:
a magnetic device positionable within a body of a patient; and
a surgical instrument including a magnetic detection device operably disposed thereon to detect the magnetic device, the surgical instrument positionable within the body of the patient and configured to interact with tissue of interest within the patient.

2. A system according to claim 1, wherein the magnetic device is selected from the group consisting of a magnetic element in the form of one of a clip and tag, stent, staple, silvered sutures, injectable solution including a viscous base material with magnetic particles, and powered chip configured to emit one of an electromagnetic and magnetic field.

3. A system according to claim 1, wherein the magnetic device is positioned within the body of a patient via one of an endoscope and being ingested.

4. A system according to claim 1, wherein the magnetic device is operably coupled to a surgical mesh that includes a helical tack thereon for attaching the surgical mesh including the magnetic device to tissue adjacent the tissue of interest.

5. A system according to claim 2, wherein the powered chip is one of an active RFID device and a passive RFID device.

6. A system according to claim 5, wherein the magnetic detection device on the surgical instrument includes at least one RFID detector thereon that is configured to detect one of the active and passive RFID devices.

7. A system according to claim 2, wherein the injectable solution including a viscous base material with magnetic particles is India ink with one of magnetic and ferrous particles suspended therein.

8. A system according to claim 1, wherein the magnetic detection device on the surgical instrument includes at least two coils that are positioned in close proximity to one another and rigidly fixed with respect to one another, wherein in a non-magnetic media the at least two coils have a stable mutual coupling of their magnetic fields.

9. A system according to claim 8, wherein, when the at least two coils are positioned adjacent the magnetic device, at least one indication is provided to an end user, wherein the at least one indication includes one of an audio and a visual indicator.

10. A system according to claim 1, wherein the magnetic detection device on the surgical instrument includes a single coil and when the coil is positioned adjacent the magnetic device, at least one indication is provided to an end user, wherein the at least one indication includes one of an audio and a visual indicator.

11. A system according to claim 1, wherein the tissue of interest is part of a bowel, liver, lung or fibroids.

12. A system according to claim 11, wherein the magnetic device is configured to penetrate through a mucosa and into a submucosal space of a bowel wall to prevent migration of the magnetic device.

13. A system according to claim 1, wherein the magnetic detection device is configured to create software-coupled imaging information to a monitor in real-time.

14. A system according to claim 1, further including a robotic arm having a sensor thereon that is configured to produce a signal as a result of the sensor coming into proximity of the magnetic device, the signal being representative of the position of the magnetic device with respect to the sensor.

15. A system according to claim 1, wherein the surgical instrument is a transverse anastomosis stapler that is configured to staple tissue and, subsequently, sever the stapled tissue.

16. A system for navigating a surgical instrument adjacent tissue of interest, comprising:
at least one magnetic device positionable within a body of a patient and attachable to tissue adjacent the tissue of interest, the magnetic device emitting a magnetic field;
a surgical instrument including a magnetic detection device operably disposed thereon and configured to detect the magnetic field emitted by the magnetic device, the surgical instrument configured to remove tissue of interest within the patient; and
a monitor in operable communication with at least one of the magnetic device and surgical instrument, the monitor configured to provide at least one of a visual and audio indication of the proximity of the surgical instrument with respect to the magnetic device.

17. A system according to claim 16, wherein the magnetic device is selected from the group consisting of a magnetic element in the form one of a clip and tag, stent, staple, silvered sutures, injectable solution including a viscous base material with magnetic particles, and powered chip configured to emit one of an electromagnetic and magnetic field.

18. A system according to claim 16, wherein the magnetic device is positioned within the body of a patient via one of an endoscope and being ingested.

19. A system according to claim 16, wherein the magnetic detection device on the surgical instrument includes at least two coils that are positioned in close proximity to one another and are rigidly fixed with respect one another, wherein in a non-magnetic media the at least two coils have a stable mutual coupling of their magnetic fields, wherein when the at least two coils are positioned adjacent the magnetic device at least one indication is provided to the monitor, wherein the at least one indication includes one of an audio and a visual indicator.

20. A system according to claim 16, wherein the magnetic detection device on the surgical instrument includes a single coil and when the coil is positioned adjacent the magnetic device, at least one indication is provided to an end user, wherein the at least one indication includes one of an audio and a visual indicator.
